(19) United States
(12) Patent Application Publication
(10) Pub. No.: US 2020/0147070 A1
(43) Pub. Date: May 14, 2020

(54) INDUCING PHOSPHOLIPIDOSIS FOR ENHANCING THERAPEUTIC EFFICACY

(71) Applicant: Nextcea Inc., Woburn, MA (US)

(72) Inventor: Frank Hsieh, Woburn, MA (US)

(21) Appl. No.: 16/614,152

(22) PCT Filed: May 16, 2018
(86) PCT No.: PCT/US2018/032885

§ 371 Date: Nov. 15, 2019

Related U.S. Application Data
(60) Provisional application No. 62/507,537, filed on May 17, 2017.

(51) Int. Cl. A61K 31/47; G01N 33/92; A61K 31/343; A61K 31/138

(52) U.S. Cl. CPC A61K 31/47; A61K 31/138; A61K 31/343; G01N 33/92

(57) ABSTRACT

A method of enhancing the efficacy of a therapeutic compound, the method comprising administering an effective amount of a phospholipidosis (PL)-inducing compound (or compounds) to a patient in need of a therapeutic compound for a disorder, whereby PL is induced in the patient; and administering the therapeutic compound to the patient.

### CROSS REFERENCE TO RELATED APPLICATION

This application claims priority to U.S. Provisional Application No. 62/507,537, filed on May 17, 2017, the entire content of which is hereby incorporated by reference herein.

### BACKGROUND

Phospholipidosis (PL) is a lysosomal storage condition characterized by the accumulation of multi-lamellar (myeloid) bodies in cells and tissues. It can be induced by various natural and synthetic compounds, and is a common finding in animals and humans treated with cationic amphiphilic drugs (CADs). PL is typically reversible after the cessation of drug treatment. It can be induced at a manageable level without serious collateral drug side effects.

### SUMMARY

This invention is based, at least in part, on the unexpected discovery that PL can be used to selectively inhibit lysosomal degradation and increase drug exposure in target tissues, cells, or organs to enhance in vivo therapeutic efficacy.

In one aspect, a method is described for enhancing the efficacy of a therapeutic compound. The method includes administering an effective amount of a PL-inducing compound to a patient in need of a therapeutic compound for a disorder, whereby PL is induced in the patient; and administering the therapeutic compound to the patient.

For example, the therapeutic compound can be for treating colon cancer, breast cancer, prostate cancer, hepatocellular carcinoma, melanoma, lung cancer, glioblastoma, brain tumor, hematopoeitic malignancies, retinoblastoma, renal cell carcinoma, head and neck cancer, cervical cancer, pancreatic cancer, esophageal cancer, squama cell carcinoma, hemophila, hypercholesterolemia, inflammatory dermatoses (e.g., dermatitis, eczema, atopic dermatitis, allergic contact dermatitis, urticaria, necrotizing vasculitis, cutaneous vasculitis, hypersensitivity vasculitis, eosinophilic myositis, polymyositis, dermatomyositis, and eosinophilic fasciitis), inflammatory bowel diseases (e.g., Crohn's disease and ulcerative colitis), acute respiratory distress syndrome, fulminant hepatitis, hypersensitivity lung diseases (e.g., hypersensitivity pneumonitis, eosinophilic pneumonia, delayed-type hypersensitivity, interstitial lung disease or ILD, idiopathic pulmonary fibrosis, and ILD associated with rheumatoid arthritis), asthma, and allergic rhinitis, autoimmune diseases (e.g., rheumatoid arthritis, psoriatic arthritis, systemic lupus erythematosus, myasthenia gravis, juvenile onset diabetes, glomerulonephritis, autoimmune throiditis, ankylosing spondylitis, systemic sclerosis, and multiple sclerosis), acute and chronic inflammatory diseases (e.g., systemic anaphylaxia or hypersensitivity responses, drug allergies, insect sting allergies, allograft rejection, and graft-versus-host disease), Sjogren's syndrome, human immunodeficiency virus infection, tumor metastasis, bronchitis, cystic fibrosis, chronic obstructive lung disease, kidney diseases (e.g., kidney cancer, nephritis, and nephropathy), and neurological diseases (e.g., Alzheimer's disease, Parkinson's disease, Huntington's disease, amyotrophic lateral sclerosis).

In some embodiments, the PL-inducing compound induces PL in a tissue, organ, or cell affected by the disorder or intended to be targeted by the therapeutic compound. In other embodiments, the PL-inducing compound induces PL in a tissue, organ, or cell not affected by the disorder or not intended to be targeted by the therapeutic compound. In some embodiments, the PL-inducing compound induces multi-organ PL.

In some embodiments, administration of the PL-inducing compound decreases lysosomal degradation of the therapeutic compound. In some embodiments, administration of the PL-inducing compound increases the concentration of the therapeutic compound in a tissue, organ, or cell affected by the disorder or intended to be targeted by the therapeutic compound. In some embodiments, administration of the PL-inducing compound reduces the concentration of the therapeutic compound in a tissue, organ, or cell not affected by the disorder or not intended to be targeted by the therapeutic compound.

In some embodiments, the PL-inducing compound and the therapeutic compound are administered at the same time or about the same time before PL is induced in the patient. In some embodiments, the therapeutic compound is administered only after PL is induced in the patient. In some embodiments, the patient is treated with the therapeutic compound for a treatment period and PL is induced for a portion or over the entire duration of the treatment period.

The method can further include monitoring the occurrence, progress, or reversibility of PL in the patient. In some embodiments, the monitoring step includes detecting the levels of one or more biomarkers in a biological sample obtained from the patient, the one or more biomarkers being selected from the group consisting of 2,2′ di-22:6-BMP, 3,2′ di-22:6-BMP, 2,3′ di-22:6-BMP, 3,3′ di-22:6-BMP, di-18:1-BMP, di-18:2-BMP, 18:1/18:2-BMP, 18:1/22:6-BMP, 18:2/22:6-BMP, di-22:6-PG, di-18:1-PG, di-18:2-PG, 18:1/18:2-PG, 18:1/22:6-PG, 18:2/22:6-PG, mono-22:6-BMP, mono-18:1-BMP, and mono-18:2-BMP. The monitoring step can further include detecting the levels of one or more additional species of BMP, PG, or mono-BMP, or total BMP. In some embodiments, the therapeutic compound is administered after an elevated level of the biomarker, as compared to a control level, is detected in the biological sample.

The therapeutic compound can be a biological drug, e.g., an antibody, antibody drug conjugate (ADC), protein, peptide, peptidomimetic, peptoid, RNA, DNA, siRNA, miRNA, or RNA aptamer. In some embodiments, the therapeutic compound is a small molecule drug.

In some embodiments, the PL-inducing compound is selected from the group consisting of ambroxol (metabolite of bromhexine), amikacin, amiodarone, amitriptyline, aripiprazole, atorvastatin, azithromycin, bedaquiline (TMC207, R207910), bepotastine, bromopheniramine, busulfan, chlorcyclizine, chloropromazine, chlorpheniramine (chlorphenamine), chloroquine, citalopram, clarithromycin, clindamycin, cloforex (prodrug of chlrophentermine), clomipramine, clozapine, crizotinib, cyclizine, dronedarone, SR33589, duloxetine, erythromycin, escitalopram, everolimus, RAD001, fluoxetine, gentamicin, haloperidol, homochlorcyclizine, hydroxychloroquine, hydroxyzine, imipramine (G 22355, melipramine), indormamin, iprindole (pramindole), ketotifen, kevodopa (L-DOPA), kaprotiline, meclizine, memantine, nortriptyline, noxiptiline (noxiptyline or dibenzoxine), paroxetine, pentamidine, pheniramine, phentermine, posaconazole, promethazine, quinacrine (mepacrine), rapamycin, rosuvastatin, sapropterin (tetrahydrobiopterin), simvastatin, tamoxifen, telithromycin, tobramycin, trifluperazine, trimeprazine (alimenazine), trimethoprim, tunicamycin, vandetanib, verenicline, zonisamide, poloxamers (pluronics, synperonics, and kolliphor), total parenteral nutrition (TPN) solutions, steroid hormones, and oxysterols. One or more PL-inducing compounds can be used to induce PL in a subject.

The therapeutic compound can be any of the PL-inducing compounds listed above or selected from the group consisting of abogovomab, abciximab, abagovomab, abciximab, abrilumab, actoxumab, adalimumab, adecatumumab, aducanumab, afelimomab, afutuzumab, alacizumab pegol, aLD518, alemtuzumab, alirocumab, altumomab pentetate, amatuximab, anatumomab mafenatox, anetumab ravtansine, anifrolumab, anrukinzumab, apolizumab, arcitumomab, ascrinvacumab, aselizumab, atezolizumab, atinumab, atlizumab (tocilizumab), atorolimumab, bapineuzumab, basiliximab, bavituximab, bectumomab, begelomab, belimumab, benralizumab, bertilimumab, besilesomab, bevacizumab, bezlotoxumab, biciromab, bimagrumab, bimekizumab, bivatuzumab mertansine, blinatumomab, blosozumab, bococizumab, brentuximab vedotin, briakinumab, brodalumab, brolucizumab, brontictuzumab, canakinumab, cantuzumab mertansine, cantuzumab ravtansine, caplacizumab, capromab pendetide, carlumab, catumaxomab, cBR96-doxorubicin immunoconjugate, cedelizumab, certolizumab pegol, cetuximab, citatuzumab bogatox, cixutumumab, clazakizumab, clenoliximab, clivatuzumab tetraxetan, codrituzumab, coltuximab ravtansine, conatumumab, concizumab, cR6261, crenezumab, dacetuzumab, daclizumab, dalotuzumab, dapirolizumab pegol, daratumumab, dectrekumab, demcizumab, denintuzumab mafodotin, denosumab, derlotuximab biotin, detumomab, dinutuximab, diridavumab, dorlimomab aritox, drozitumab, duligotumab, dupilumab, durvalumab, dusigitumab, ecromeximab, eculizumab, edobacomab, edrecolomab, efalizumab, efungumab, eldelumab, elgemtumab, elotuzumab, elsilimomab, emactuzumab, emibetuzumab, enavatuzumab, enfortumab vedotin, enlimomab pegol, enoblituzumab, enokizumab, enoticumab, ensituximab, epitumomab cituxetan, epratuzumab, erlizumab, ertumaxomab, etaracizumab, etrolizumab, evinacumab, evolocumab, exbivirumab, fanolesomab, faralimomab, farletuzumab, fasinumab, FBTA05, felvizumab, fezakinumab, ficlatuzumab, figitumumab, firivumab, flanvotumab, fletikumab, fontolizumab, foralumab, foravirumab, fresolimumab, fulranumab, futuximab, galiximab, ganitumab, gantenerumab, gavilimomab, gemtuzumab ozogamicin, gevokizumab girentuximab, glembatumumab vedotin, golimumab, gomiliximab, guselkumab, ibalizumab, ibritumomab tiuxetan, icrucumab, idarucizumab, igovomab, IMAB362, imalumab, imciromab, imgatuzumab, inclacumab, indatuximab ravtansine, indusatumab vedotin, infliximab, inolimomab, inotuzumab ozogamicin, intetumumab, ipilimumab, iratumumab, isatuximab, itolizumab, ixekizumab, keliximab, labetuzumab, lambrolizumab, lampalizumab, lebrikizumab, lemalesomab, lenzilumab, lerdelimumab, lexatumumab, libivirumab, lifastuzumab vedotin, ligelizumab, lilotomab satetraxetan, lintuzumab, lirilumab, lodelcizumab, lokivetmab, lorvotuzumab mertansine, lucatumumab, lulizumab pegol, lumiliximab, lumretuzumab, mapatumumab, margetuximab, maslimomab, matuzumab, mavrilimumab, mepolizumab, metelimumab, milatuzumab, minretumomab, mirvetuximab soravtansine, mitumomab, mogamulizumab, morolimumab, motavizumab, moxetumomab pasudotox, muromonab-CD3, nacolomab tafenatox, namilumab, naptumomab estafenatox, narnatumab, natalizumab, nebacumab, necitumumab, nemolizumab, nerelimomab, nesvacumab, nimotuzumab, nivolumab, nofetumomab merpentan, obiltoxaximab, obinutuzumab, ocaratuzumab, ocrelizumab, odulimomab, ofatumumab, olaratumab, olokizumab, omalizumab, onartuzumab, ontuxizumab, opicinumab, oportuzumab monatox, oregovomab, orticumab, otelixizumab, otlertuzumab, oxelumab, ozanezumab, ozoralizumab, pagibaximab, palivizumab, panitumumab, pankomab, panobacumab, parsatuzumab, pascolizumab, pasotuxizumab, pateclizumab, patritumab, pembrolizumab, pemtumomab, perakizumab, pertuzumab, pexelizumab, pidilizumab, pinatuzumab vedotin, pintumomab, placulumab, polatuzumab vedotin, ponezumab, priliximab, pritoxaximab, pritumumab, PRO 140, quilizumab, racotumomab, radretumab, rafivirumab, ralpancizumab, ramucirumab, ranibizumab, raxibacumab, refanezumab, regavirumab, reslizumab, rilotumumab, rinucumab, rituximab, robatumumab, roledumab, romosozumab, rontalizumab, rovelizumab, ruplizumab, sacituzumab govitecan, samalizumab, sarilumab, satumomab pendetide, secukinumab, seribantumab, setoxaximab, sevirumab, SGN-CD19A, SGN-CD33A, sibrotuzumab, sifalimumab, siltuximab, simtuzumab, siplizumab, sirukumab, sofituzumab vedotin, solanezumab, solitomab, sonepcizumab, sontuzumab, stamulumab, sulesomab, suvizumab, tabalumab, tacatuzumab tetraxetan, tadocizumab, talizumab, tanezumab, taplitumomab paptox, tarextumab, tefibazumab, telimomab aritox, tenatumomab, teneliximab, teplizumab, teprotumumab, tesidolumab, tetulomab, TGN1412, ticilimumab (tremelimumab), tigatuzumab, tildrakizumab, TNX-650, tocilizumab (atlizumab), toralizumab, tosatoxumab, tositumomab, tovetumab, tralokinumab, trastuzumab, trastuzumab emtansine, TRBS07, tregalizumab, tremelimumab, trevogrumab, tucotuzumab, celmoleukin, tuvirumab, ublituximab, ulocuplumab, urelumab, urtoxazumab, ustekinumab, vandortuzumab vedotin, vantictumab, vanucizumab, vapaliximab, varlilumab, vatelizumab, vedolizumab, veltuzumab, vepalimomab, vesencumab, visilizumab, volociximab, vorsetuzumab mafodotin, votumumab, zalutumumab, zanolimumab, zatuximab, ziralimumab, zolimomab aritox, etanercept, insulin glargine, pegfilgrastim, salmon calcitonin, cyclosporine, octreotide, liraglutide, bivalirudin, desmoprossin, interferon beta-1a, interferon beta-1a, patisiran (ALN-TTR02), revusiran (ALN-TTRsc), fitusiran (ALN-AT3), ALN-CCr, ALN-AS1, ALN-PCSsc, ALN-VSP02, siRNA-EphA2-DOPC, Atu027, TKM-0880301, TKM-100201, ALN-RSV01, PRO-040201, ALN-TTR02, CALAA-01, TD101, AGN211745, QPI-1007, I5NP, PF-655 (PF-04523655), siG12D LODER, bevasiranib, SYL1001, SYL040012, SYL040012, CEQ508, RXi-109, ARC-520, mipomersen, trabedersen (AP12009), fomivirsen, iomitapide, brentuximab (Adcetris), and ado-trastuzamab emtanisine (Kadcyla).

In another aspect, described herein is a pharmaceutical composition containing a PL-inducing compound and a therapeutic compound for a disorder. The PL-inducing compound and the therapeutic compounds can be selected from the compounds listed above.

In some embodiments, the composition is a controlled-released formulation (e.g. delayed- or extended-release formulation). In some embodiment, the formulation is formulated to release the PL-inducing compound and the therapeutic compound simultaneously or at different rates or times. In some embodiments, the PL-inducing compound is released before the therapeutic compound. The composition can be a target-released formulation. In some embodiments, the composition is an implant or transdermal device.

The details of one or more embodiments of the invention are set forth in the accompanying drawings and the description below. Other features, objects, and advantages of the invention will be apparent from the description and drawings, and from the claims.

### DESCRIPTION OF DRAWINGS

FIG. 1 is a diagram showing structures of bis(monoacylglycerol) phosphate (BMP) isoforms.

FIG. 2A is a diagram showing an exemplary therapeutic PL regimen to inhibit the lysosomal degradation of biological drugs in disease cells or tissues to improve in vivo efficacy.

FIG. 2B is a diagram showing an exemplary therapeutic PL regimen to decrease the release of active toxins or drugs from ADCs in healthy/non-targeted cells or tissues to improve in vivo efficacy.

FIG. 3 is a set of electron micrographs depicting the accumulation of lysosomal myeloid bodies, indicative of PL, in rat tissues. Sprague-Dawley rats received once daily oral (PO) co-administration of bedaquiline (60 mg/kg/day) and citalopram (70 mg/kg/day) for 2 weeks.

FIG. 4 is a set of graphs showing drug concentrations in tissues of rats with PL induced by co-administration of bedaquiline (60 mg/kg/day) and citalopram (70 mg/kg/day) for 2-weeks.

FIG. 5 is a set of graphs showing di-22:6-BMP concentrations (including all isoforms) in tissues and urine of rats with PL induced by co-administration of bedaquiline (60 mg/kg/day) and citalopram (70 mg/kg/day) for 2-weeks.

FIG. 6 is a set of graphs showing that drug-induced PL decreased lysosomal degradation of 14-3-3 proteins. Levels of 14-3-3 gamma proteins were higher in the livers of rats with drug-induced PL compared to vehicle/controls. Urine di-22:6 BMP was used to monitor PL.

FIG. 7 is a set of graphs showing that PL decreased the activity of cathepsin-B, a lysosomal protease in rat tissues. Samples that contain cathepsin-B cleaved the synthetic substrate RR-AFC to release free AFC.

FIG. 8 is a set of graphs showing that PL-induction increased drug tissue concentrations to enhance in vivo drug efficacy.

FIG. 9 is a graph showing di-22:6-BMP concentrations (including all isoforms) in tissues and urine of rats treated with amiodarone alone (150 mg/kg/day) and co-administered with fluoxetine (10 mg/kg/day) for 2-weeks.

FIG. 10 is a set of pie charts showing that PL increased the efficacy of a drug targeted to a particular tissue by increasing drug exposure in that tissue. Sprague-Dawley rats were treated with amiodarone alone (150 mg/kg/day) or co-administered with fluoxetine (10 mg/kg/day) for 2 weeks. Administration with fluoxetine shifted amiodarone toward the target organ (heart) to increase efficacy and away from lung to reduce amiodarone-induced pulmonary toxicity.

### DETAILED DESCRIPTION

This disclosure is based, at least in part, on the unexpected discovery that PL can be used to preferentially inhibit lysosomal degradation and increase drug exposure in target tissues, organs or cells and thereby enhance therapeutic efficacy.

### Phospholipidosis

PL is induced by various natural and synthetic agents, including widely used marketed drugs. It is characterized by the excessive accumulation of multi-lamellar (myeloid) and/or zebra bodies in the lysosomes of affected cells. Some tissues, such as lung, kidney, and skin, normally contain myeloid bodies which represent main storage sites for undigested and secreted materials. See e.g., Schmitz and Müller, Journal of Lipid Research (1991) 32:1539-1570. Many cell and tissue types are reported as being susceptible to drug-induced PL (e.g., liver, lung, kidney, and heart). See e.g., Hruban, Environmental Health Perspectives (1984) 55:53-76. In this context, multi-lamellar bodies over accumulate in various tissues and serve as repositories for drugs, drug metabolites, undigested drug-phospholipid complexes, and other undigested cellular materials. The pattern of PL is drug, species, and tissue dependent. It is typically reversible after the discontinuation of drug treatment. At a manageable level, PL can be manifested in the absence of or without serious drug side effects. See e.g., Cartwright et al., Toxicologic Pathology (2009) 37:902-910.

### Biomarkers for Phospholipidosis

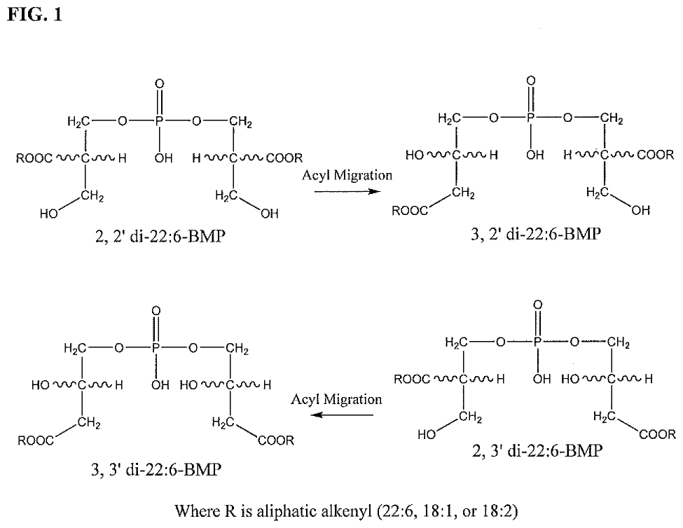

Myeloid bodies are rich in bis(monoacylglycerol)phosphate (BMP), a phospholipid uniquely found in lysosomes and late endosomes. BMP can theoretically exist in four geometrical isoforms. See FIG. 1. Molecular species of BMP with different fatty acid chains (e.g. 22:6, 18:1, and 18:2 fatty acids) correlate differentially with the patterns of tissue PL induced by various drugs. See US2010/0267061. An increase in BMP occurs in PL because of its important roles in late endosomal/lysosomal degradation pathways. BMP isoforms are sensitive and specific biomarkers of tissue PL. They can be monitored in the plasma, serum, and urine. BMP isoforms that can be used to monitor PL include 2,2′ di-22:6-BMP, 3,2′ di-22:6-BMP, 2,3′ di-22:6-BMP, 3,3′ di-22:6-BMP, di-18:1-BMP, di-18:2-BMP, 18:1/18:2-BMP, 18:1/22:6-BMP, 18:2/22:6-BMP, di-22:6-PG, di-18:1-PG, di-18:2-PG, 18:1/18:2-PG, 18:1/22:6-PG, 18:2/22:6-PG, mono-22:6-BMP, mono-18:1-BMP, and mono-18:2-BMP. Techniques for detecting and quantifying BMP isoforms are known in the art. See, e.g., US2010/0267061.

### Lysosomal Degradation of Bio-Therapeutic Drugs

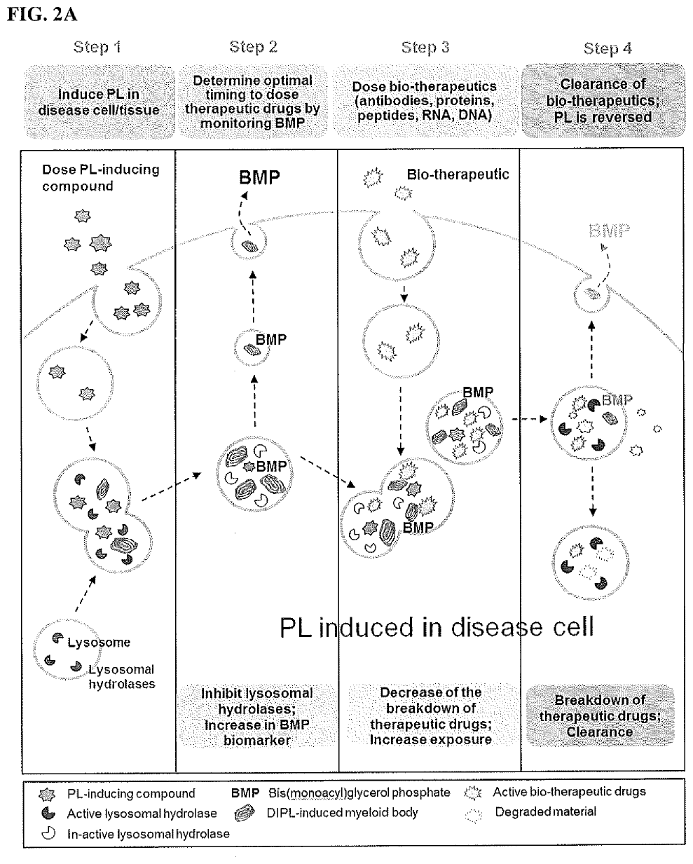

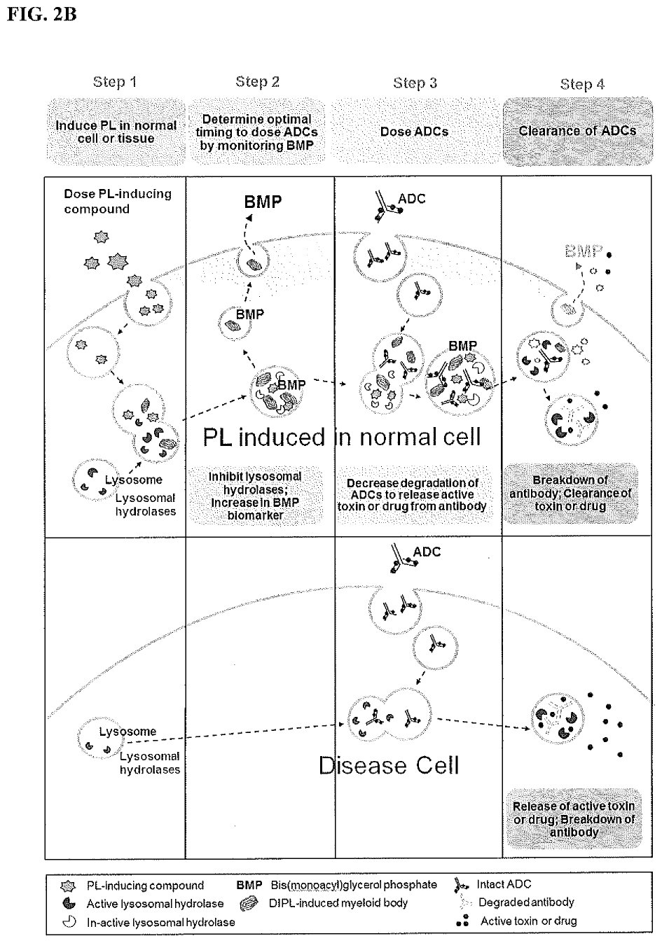

Lysosomes are responsible for the breakdown of macromolecules (e.g. lipids, proteins, carbohydrates, DNA, and RNA) derived from the extracellular space through endocytosis or phagocytosis, and from the cytoplasm through autophagy. See FIG. 2A. Lysosomes contain resident hydrolases that permit the collective degradation of all types of macromolecules, including biologics (e.g. antibodies, proteins, peptides, and nucleic acids). The acidic environment of the lysosomal lumen (pH 4.5-5.0) facilitates the degradation process by loosening the structures of macromolecules and is optimal for the activities of lysosomal hydrolases. See e.g., Appleqvist et al., Annals of Clinical and Laboratory Science (2012) 42(3):231-242; and Zhang et al., Acta Biochim Biophys Sin (2009) 41: 437-445.

Lysosomal Release of Toxins from Antibody-Drug Conjugates

An antibody-drug conjugate (ADC) consists of an antibody conjugated to a toxin or drug via a cleavable linker. ADCs are designed to bind specific proteins (e.g., receptors) expressed on the surface of cells (e.g. cancer cells) intended for treatment. After entering the cell, the linker is cleaved within the lysosome, thereby releasing the drug or toxin within the targeted cell. See FIG. 2B. The toxin or drug remains inactive while conjugated to the antibody.

For example, a peptide linker can be cleaved by lysosomal proteases such as cathepsin B. Alternatively, when the ADC is in the target cell, the antibody is degraded and the toxin is released within the lysosome.

### Phospholipidosis for Enhancing Drug Efficacy

Described herein are methods for enhancing the efficacy of therapeutic compounds (e.g., biological drugs and traditional small molecule drugs) by inducing PL. PL can inhibit the lysosomal degradation of drugs or the release of drugs from ADCs, thereby enhancing therapeutic efficacy in targeted cells, tissue or organ, or protect non-targeted cells, tissues, or organ from exposure to the drugs. See FIGS. 2A and 2B. PL can also be used to preferentially concentrate traditional small molecule drugs in target cells, tissues, or organs, and spare exposure to non-targeted cells/tissues.

The PL-inducing compound used in the treatment methods can be one that induces PL in a specific tissue, organ and/or cell type intended to be targeted by the therapeutic compounds. Alternatively, it can be one that induces PL systemically or in multiple tissues, organs or cell types. PL can be induced by one or a combination of compounds to optimize the PL conditions to optimally inhibit lysosomal enzymatic activity for a particular therapeutic compound.

To practice the treatment methods described herein, the above-described BMP biomarkers can be used to monitor the occurrence, progress, and reversibility of PL in humans or test animals. A subject has PL if the level(s) of the biomarker(s) in a biological sample obtained from the subject are at or above their corresponding control levels. A control level can be the level found in a biological sample from a control subject with PL (e.g., induced by a compound) or biological samples from a control group of subjects with PL. In some instances, the control level can be the level in a biological sample obtained from the subject to be treated with the therapeutic compound before PL is induced in the subject.

The biological sample can be a bodily fluid sample, including but not limited to whole blood, plasma, serum, urine, and saliva. It can also be a cell, cell fraction, or a cell culture. The sample can also be a whole tissue, tissue slice, or tissue fraction. The sample can also be isolated endocytic vesicles, such as endosomes, lysosomes, and exosomes derived from cells and tissues.

The methods can be used to improve the efficacy of drugs used to treat various diseases, disorders, conditions and syndromes such as cancer (e.g. lung cancer, breast cancer, prostate cancer, colon cancer) and neurological diseases (e g Alzheimer's disease, Parkinson's disease).

### I. Drugs Subjected to Lysosomal Degradation

A number of drugs, such as antibodies, proteins, peptides, and nucleic acid drugs, are internalized into cells by endocytosis and then degraded by lysosomal enzymes. See FIG. 2A.

PL-inducing compounds can directly and/or indirectly (i.e., through the accumulation of undigested materials or change in lysosomal pH) inhibit lysosomal enzyme activities. The induction of PL by such compounds can be used to prevent the degradation of therapeutic drugs (e.g. antibodies, proteins, peptides, peptidomimetics, peptoids, RNA, DNA, siRNA, miRNA, or RNA aptamer) by lysosomal hydrolases thereby increasing their in vivo efficacy. See FIG. 2A. In particular, PL can be induced in diseased or target cells, tissues, or organs to more specifically inhibit lysosomal degradation of the drugs in those cells, tissues, or organs.

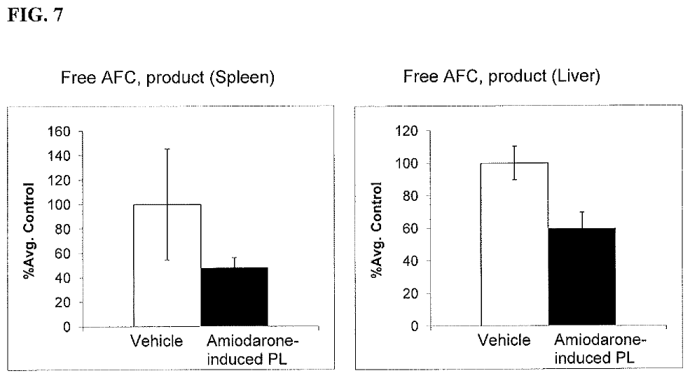

PL has been shown to inhibit lysosomal proteases such as cathepsin B. For example, the PL-inducing chloroquine inhibited cathepsin B1 activities. See, e.g., Wibo and Poole, The Journal of Cell Biology (1974) 63:430-440. As described below, PL induced by the antibiotic bedaquiline unexpectedly slowed the lysosomal degradation of proteins in rat liver. See FIG. 6. PL induced by amiodarone slowed the activity of cathepsin B in rat liver and spleen. See FIG. 7.

To practice the treatment method, PL can be induced in a subject to be treated with a therapeutic compound that is subjected to lysosomal degradation. PL can be induced at the same time or after the therapeutic compound is administered. The biomarkers described herein can be used to monitor PL in the subject. For example, the biomarkers can be used to determine when the therapeutic drug should be administered. Once PL is induced, it is maintained or intensified to optimize therapy until the desired therapeutic effect is achieved or the therapeutic treatment is completed. PL is discontinued or reversed (e.g., by discontinuing the PL treatment) after completion of the therapeutic treatment.

### II. Drugs Activated by Lysosomal Processing

A number of drugs, such as ADCs, are internalized into cells by endocytosis and activated through lysosomal processing. See FIG. 2B. A PL-inducing compound can be administered with an ADC cleavable by a lysosomal protease to inhibit release of the drug in a non-target organ, cell, or tissue. The drug is thereby released preferentially within the target organ, cell, or tissue to enhance drug efficacy. See FIG. 2B.

More specifically, a patient in need of an ADC is treated with the ADC and one or more PL-inducing compounds to induce PL and inhibit lysosomal protease activities in the cell, organ or tissue not intended to be targeted by the ADC. PL can be induced at the same time or after the ADC is administered. The biomarkers described herein can be used to monitor PL in the subject and determine when the ADC should be administered. For example, using the method, healthy cells can be protected from an ADC intended to target and kill cancer cells.

PL is maintained until the therapeutic effect or an effective concentration of the ADC has been achieved in the target cell, tissue, or organ. The PL-inducing treatment is then discontinued after completion of the therapeutic treatment.

### III. Drugs Targeted to Tissues, Organs or Cells by Phospholipidosis

Therapeutic efficacy can also be enhanced by using PL to increase the concentrations of small molecule drugs in target cells, tissues or organs and to preferentially distribute them, thereby increasing drug exposure in the target cells, tissues or organs.

Thus, a patient in need of a therapeutic treatment can be administered with a small molecule drug together with one or more PL-inducing compounds that induce PL in the intended target cell, tissue or organ of the drug. The drug is circulated and concentrated within myeloid bodies and then released in a controlled manner (by modulating PL using PL-inducing compounds as monitored by biomarkers) within the target tissue, cell or organ where the drug produces a therapeutic effect.

PL is maintained until the effect or an effective amount of the therapeutic molecule has been achieved in the target cell, tissue or organ. PL is then reversed after completion of the therapeutic treatment.

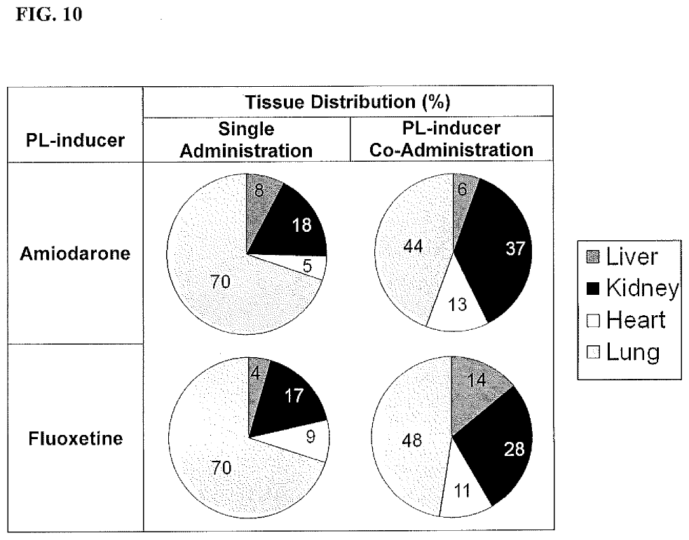

For example, as described below, an increased concentration of fluoxetine (Prozac) occurred in tissues, including the target tissue brain, during co-administration with the PL-inducing compound amiodarone as compared with fluoxetine alone. Fluoxetine uptake in tissues increased with tissue di-22:6-BMP concentrations. See FIG. 10

In another example, the method can be used to enhance the efficacy of polymer drug conjugates and biologics that have been modified to extend their in vivo half-lives (e.g., pegylated proteins, pegylated peptides, and fusion proteins).

### Pharmaceutical Compositions

To practice the above-described treatment methods, a pharmaceutical composition containing one or more PL-inducing compounds and a therapeutic drug can be used. The composition can be formulated as a controlled-release (e.g., timed-release, delayed-release, or extended-release) formulation.

The release profile of the formulation can be designed to enhance the efficacy of a therapeutic drug as described above. For example, different therapeutic regimens or controlled-release formulations (e.g., different combinations of PL-inducing compounds and therapeutic drugs, different release profiles, or different orders/timings of administration) can be tested in test human subjects or animal models.

For example, the formulation can be formulated to release a PL-inducing compound and a therapeutic drug at the same time. In another example, the formulation can be formulated to first release a PL-inducing compound to induce PL in the patient and then release a therapeutic drug. The release of either or both the PL-inducing compound and the therapeutic drug can be sustained or stopped as required to achieve the desired result. The PL biomarkers can be used to determine and monitor the optimal dosing time for the therapeutic compound. Methods and materials for designing and producing controlled-release formulations are known in the art.

The specific examples below are to be construed as merely illustrative, and not limitative of the remainder of the disclosure in any way whatsoever. Without further elaboration, it is believed that one skilled in the art can, based on the description herein, utilize the present invention to its fullest extent. All publications cited herein are hereby incorporated by reference in their entirety. Further, any mechanism proposed below does not in any way restrict the scope of the claimed invention.

### Example 1

Sprague-Dawley rats were co-administered bedaquiline (60 mg/kg/day) and citalopram (70 mg/kg/day) in a 2-week repeat dose study. Tissue sections (liver, kidney, and heart) were collected at 2 weeks for electron microscopic examination and PL biomarker/proteomic evaluation.

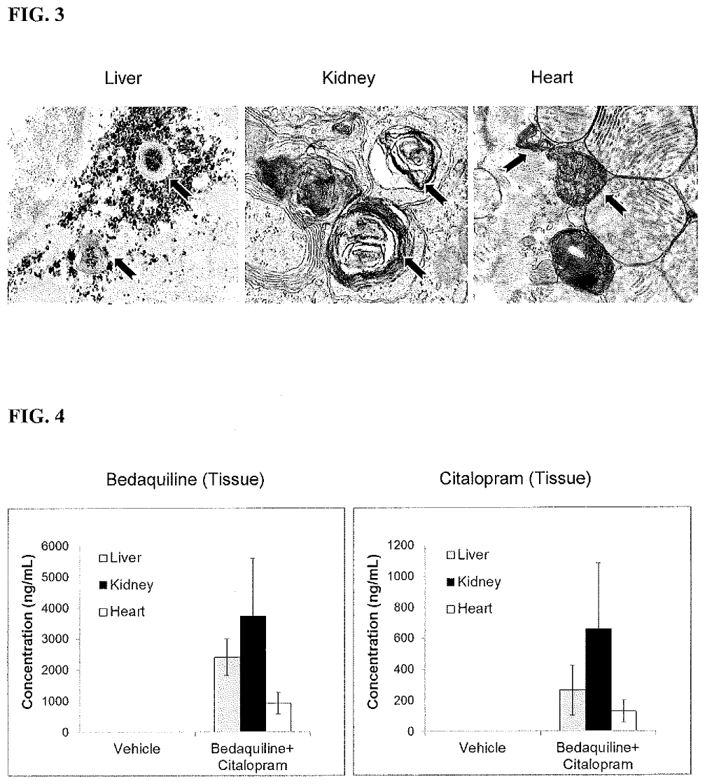

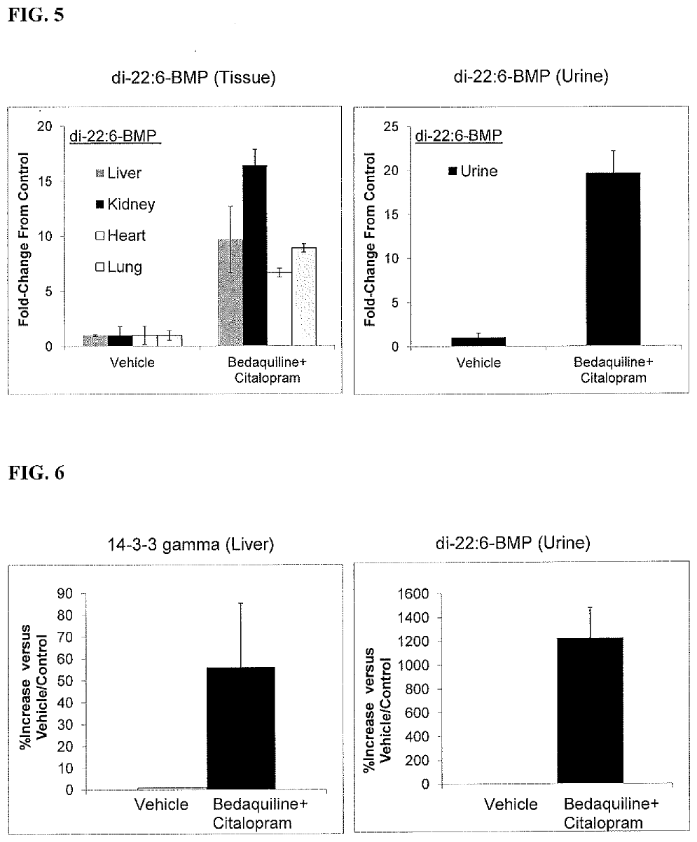

Representative electron micrographs are shown in FIG. 3. A treatment-dependent increase in multi-lamellar lysosomal inclusions and electron dense deposits was observed in liver, kidney, and heart, indicative of drug-induced PL. Concentrations of bedaquiline and citalopram in tissues are shown in FIG. 4. A corresponding increase in the biomarker di-22:6-BMP was observed with drug concentrations and PL in rat tissues and urine. See FIG. 5.

In rats with PL, the levels of 14-3-3 gamma protein were unexpectedly higher in liver compared to the vehicle/control group. See FIG. 6. 14-3-3 proteins are cytosolic proteins involved in regulating various intracellular signaling, cell cycling, apoptosis, and transcription regulation processes. See, e.g., Tzivion et al., Oncogene (2001) 20:6331-6338. 14-3-3 proteins are reported substrates of cathepsins (D, L, S, and B), a family of lysosomal proteases. See, e.g., Appelqvist et al., Annals of Clinical and Laboratory Science (2012) 42(3):231-242; and Završnik et al., Biochemical and Biophysical Research Communications (2015) 465(2):213-217.

It is proposed that the unexpected increase in liver 14-3-3 gamma was due to PL inhibition of lysosomal protease activity either within the lysosomal or release to the cytosol. Urine di-22:6-BMP isoforms can be used to monitor drug concentrations to induce PL in specific tissues and modulate lysosomal enzyme activity.

### Example 2

Cathepsin-B is a lysosomal protease that plays an important role in intracellular proteolysis. Cathepsin-B activity was investigated in tissues from a 2-week repeat dose study of amiodarone (150 mg/kg/day) in Sprague-Dawley rats. Liver and spleen homogenates were analyzed using a fluorescence-based assay that utilized the preferred cathepsin-B substrate sequence RR labeled with amino-4-trifluoromethyl coumarin (AFC). Samples that contain cathepsin-B cleave the synthetic substrate RR-AFC to release free AFC.

The results are shown in FIG. 7. Unexpectedly, the levels of free AFC were lower in tissues with amiodarone-induced PL compared to controls, indicative of an inhibitory effect of PL on cathepsin-B activity.

### Example 3

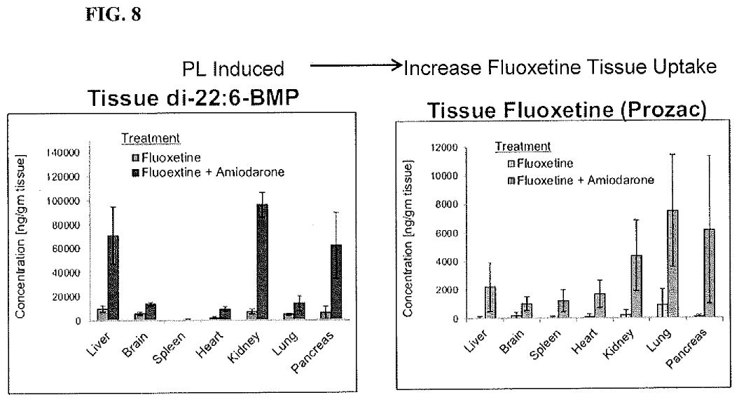

Sprague-Dawley rats were treated with fluoxetine (10 mg/kg/day) alone or co-administered with amiodarone (150 mg/kg/day) for 2 weeks. Tissues were collected at necropsy at 2 weeks. PL was induced as indicated by an increase in the di-22:6-BMP biomarker in various tissues. See FIG. 8.

Unexpectedly, fluoxetine concentrations were increased in tissues with amiodarone-induced PL compared to administration of fluoxetine alone. See FIG. 8. The results demonstrate PL can be used to enhance drug tissue uptake and thereby may enhance drug efficacy.

### Example 4

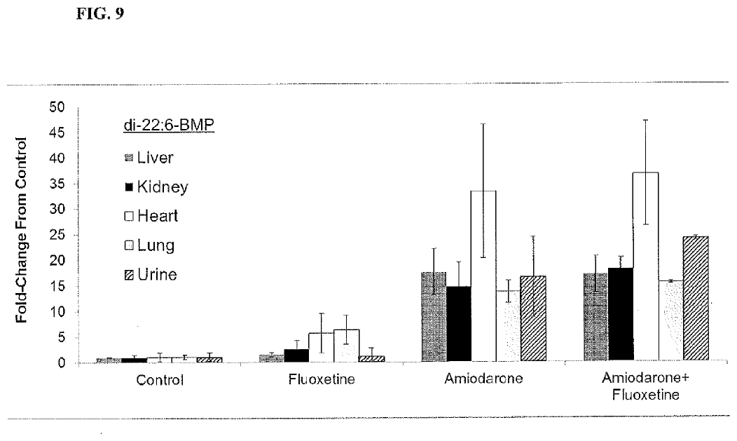

Sprague-Dawley rats were treated with amiodarone alone (150 mg/kg/day) or co-administered with fluoxetine (10 mg/kg/day) for 2 weeks. Heart, liver, kidney and lung tissues were collected at necropsy at 2 weeks. PL was induced in tissues of both treatment groups as indicated by an increase in the PL biomarker di-22:6-BMP. See FIG. 9.

### Example 5

The time to onset of action of amiodarone is often long in patients treated for arrhythmias. One reason might be a slow entry of the drug into the target organ, the heart. See, e.g. Barbieri et al., J Am Coll Cardiol (1986) July; 8(1):210-3. Co-administration of amiodarone with fluoxetine in rats (as described above) unexpectedly changed the distribution of amiodarone in rat tissues and increased the amiodarone concentration in the heart. See FIG. 10.

Additionally, amiodarone-induced pulmonary toxicity is a known serious complication of amiodarone therapy. See, e.g., Wolkove et al., Canadian Respiratory Journal (2009) 16(2):43-48. The PL-inducer therapy decreased the concentration of amiodarone in the lung tissue, thereby reducing the risk of pulmonary toxicity.

### OTHER EMBODIMENTS

All of the features disclosed in this specification may be combined in any combination. Each feature disclosed in this specification may be replaced by an alternative feature serving the same, equivalent, or similar purpose. Thus, unless expressly stated otherwise, each feature disclosed is only an example of a generic series of equivalent or similar features.

A number of embodiments of the invention have been described. Nevertheless, it will be understood that various modifications may be made without departing from the spirit and scope of the invention. Accordingly, other embodiments are within the scope of the following claims.

What is claimed is:

1. A method of enhancing the efficacy of a therapeutic compound, the method comprising:
administering an effective amount of a phospholipidosis (PL)-inducing compound to a patient in need of a therapeutic compound for a disorder, whereby PL is induced in the patient; and
administering the therapeutic compound to the patient.

2. The method of claim 1, wherein the PL-inducing compound induces PL in a tissue, organ, or cell affected by the disorder or intended to be targeted by the therapeutic compound.

3. The method of claim 1, wherein the PL-inducing compound induces PL in a tissue, organ, or cell not affected by the disorder or not intended to be targeted by the therapeutic compound.

4. The method of claim 1, wherein the PL-inducing compound induces multi-organ PL.

5. The method of claim 1, wherein administration of the PL-inducing compound decreases lysosomal degradation of the therapeutic compound.

6. The method of claim 2, wherein administration of the PL-inducing compound increases the concentration of the therapeutic compound in the tissue, organ, or cell.

7. The method of claim 3, wherein administration of the PL-inducing compound reduces concentration of the therapeutic compound in the tissue, organ, or cell.

8. The method of claim 1, wherein the PL-inducing compound and the therapeutic compound are administered at the same time or about the same time before PL is induced in the patient.

9. The method of claim 1, wherein the therapeutic compound is administered only after PL is induced in the patient.

10. The method of claim 1, further comprising monitoring the occurrence, progress, or reversibility of PL in the patient.

11. The method of claim 10, wherein the monitoring step includes detecting the levels of one or more biomarkers in a biological sample obtained from the patient, the one or more biomarkers being selected from the group consisting of 2,2′ di-22:6-BMP, 3,2′ di-22:6-BMP, 2,3′ di-22:6-BMP, 3,3′ di-22:6-BMP, di-18:1-BMP, di-18:2-BMP, 18:1/18:2-BMP, 18:1/22:6-BMP, 18:2/22:6-BMP, di-22:6-PG, di-18:1-PG, di-18:2-PG, 18:1/18:2-PG, 18:1/22:6-PG, 18:2/22:6-PG, mono-22:6-BMP, mono-18:1-BMP, and mono-18:2-BMP.

12. The method of claim 11, wherein the monitoring step further includes detecting the levels of one or more additional species of BMP, PG, or mono-BMP, or total BMP.

13. The method of claim 11, wherein the therapeutic compound is administered after an elevated level of the biomarker, as compared to a control level, is detected in the biological sample.

14. The method of claim 1, wherein the patient is administered with the therapeutic compound for a treatment period and PL is induced for a portion or over the entire duration of the treatment period.

15. The method of claim 1, wherein the therapeutic compound is a biological drug.

16. The method of claim 1, wherein the therapeutic compound is a small molecule drug.

17. A pharmaceutical composition, comprising a PL-inducing compound and a therapeutic compound for a disorder.

18. The composition of claim 17, wherein the composition is a controlled-released formulation.

19. The composition of claim 18, wherein the formulation is formulated to release the PL-inducing compound and the therapeutic compound simultaneously or at different rates or times.

20. The composition of claim 19, wherein the PL-inducing compound is released before the therapeutic compound.
